(19) United States
(12) Patent Application Publication
(10) Pub. No.: US 2024/0091161 A1
(43) Pub. Date: Mar. 21, 2024

(54) NON-ANIMAL SOFTGEL CAPSULE FORMULATIONS, METHODS OF PREPARATION, AND METHODS OF USE THEREOF

(71) Applicant: R.P. Scherer Technologies, LLC, Carson City, NV (US)

(72) Inventors: Qi Fang, St. Petersburg, FL (US); Keith Tanner, St. Petersburg, FL (US); Karunakar Sukuru, St. Petersburg, FL (US); Aristippos Gennadios, St. Petersburg, FL (US)

(21) Appl. No.: 17/767,523

(22) PCT Filed: Oct. 8, 2020
(86) PCT No.: PCT/US2020/054806

§ 371 Date: Apr. 8, 2022

Related U.S. Application Data
(60) Provisional application No. 62/912,886, filed on Oct. 9, 2019.

(51) Int. Cl. A61K 9/48; A61K 8/11; A61K 8/73; A61K 45/06

(52) U.S. Cl. CPC A61K 9/4816; A61K 8/11; A61K 8/73; A61K 45/06; A61K 2800/80

(57) ABSTRACT

Disclosed in certain embodiments is a softgel composition comprising a fill material encapsulated in a shell composition. The shell composition having a non-animal derived gelling agent and a water soluble polymer. The shell composition completely dissolving in less than 30 minutes when subject to a dissolution with a USP Apparatus II with paddies at 75 RPM in 900 ml of 0.1N HCL and deionized water at 37 degrees C.

### RELATED APPLICATIONS

This application claims priority to U.S. Provisional Patent Application No. 62/912,886, filed on Oct. 9, 2019, which is herein incorporated by reference in its entirety.

### FIELD

This disclosure relates to non-animal softgel capsule formulations. Also disclosed herein are methods of preparation of such softgel capsules and methods of use thereof.

### BACKGROUND

Encapsulating a solution or dispersion of a nutritional or pharmaceutical agent in a liquid carrier within a softgel capsule offers numerous advantages over other dosage forms such as compressed, coated or uncoated solid tablets or bulk liquid preparations. Such encapsulation of a solution or dispersion enables accurate delivery of a unit dose, which is particularly important when relatively small amounts of active ingredient must be administered. Additionally, uniformity is more difficult to achieve with a tableting process where solids must be uniformly mixed and compressed, or with incorporation of the total dose of active ingredient into a bulk liquid carrier that must be measured out prior to each oral administration.

Moreover, softgel capsules, most commonly, soft gelatin capsules, provide a dosage form which is more readily accepted by patients, since the capsules are easy to swallow and need not be flavored in order to mask the unpleasant taste of the active agent. Softgel capsules are also more easily transported by patients than bulk liquids, since only the required number of doses need to be removed from the package.

Softgel encapsulation of drugs further has the potential to improve bioavailability of pharmaceutical agents. Active ingredients are rapidly released in liquid form as soon as the shell ruptures. Complete disintegration of the capsule is not necessary for the active ingredients to become available for absorption, unlike the case of tableted compositions. Furthermore, relatively insoluble active ingredients can be dispersed in a liquid carrier to provide faster absorption.

Compared to softgel capsules made from animal derived gelatin, which takes between 5 to 15 minutes to rupture, current non-animal softgels (e.g. Vegicaps® and OptiShell® Softgel) take between 20 to 40 minutes to rupture in dissolution media. For immediate release products where fast relief is needed (e.g., analgesics, antihistamine/allergy medicines) such long dissolution time may be problematic to the patient.

Accordingly, there is a need for improved non-animal softgel capsule formulations that have shorter dissolution and/or disintegration times.

### BRIEF SUMMARY

According to various embodiments, disclosed herein is a softgel composition comprising a fill material encapsulated in a shell composition. The shell composition comprising non-animal derived gelling agent and a water soluble polymer. In certain embodiments, the shell composition completely dissolves in less than 30 minutes when subject to a dissolution test with a USP Apparatus II with paddles at 75 RPM in 900 ml of 0.1N HCL and deionized water at 37 degrees C.

According to certain embodiments, disclosed is a softgel composition comprising a fill material, that includes an active agent or a cosmetic agent, encapsulated by a shell composition, wherein the shell composition comprises: a non-animal derived gelling agent comprising carrageenan, starch, or a combination thereof; and a water soluble polymer comprising polyvinyl alcohol, pullulan gum, polylactic acid, polyvinyl alcohol-polyethylene glycol graft co-polymer, high molecular weight polyethylene glycol, povidone, a surfactant, or a combination thereof.

Also disclosed herein are various embodiments of a method of preparing a softgel composition comprising non-animal derived gelling agent and a water soluble polymer.

According to further embodiments, disclosed herein is a softgel composition comprising a fill material comprising an active agent (or a cosmetic agent) that is encapsulated by a shell composition comprising non-animal derived gelling agent and a water soluble polymer and methods of manufacture and treatment thereof.

According to yet further embodiments, disclosed herein is a method of treating a condition, treatable by the active agent(s) or cosmetic agent(s) described herein, by administering any of the softgel compositions described herein to a subject in need thereof.

### BRIEF DESCRIPTION OF THE DRAWINGS

The above and other features of the present disclosure, their nature, and various advantages will become more apparent upon consideration of the following detailed description, taken in conjunction with the accompanying drawings, in which:

FIG. 1 illustrates the release profile of active agent (e.g., ibuprofen) from a conventional non-gelatin capsule as compared to a softgel capsule according to an embodiment. The release profile is also illustrative of the dissolution profile of the shell composition of a conventional non-gelatin capsule as compared to a softgel shell composition according to an embodiment.

### DETAILED DESCRIPTION

Described herein are various embodiments of softgel compositions, e.g., softgel capsule film compositions encapsulating fill materials, methods of preparation, and methods of use thereof. It is to be understood that the invention is not limited to the details of construction or process steps set forth in the following description. The invention is capable of other embodiments and of being practiced or being carried out in a variety of ways.

Reference throughout this specification to “one embodiment,” “certain embodiments,” “one or more embodiments” or “an embodiment” means that a particular feature, structure, material, or characteristic described in connection with the embodiment is included in at least one embodiment of the invention. Thus, the appearances of the phrases such as “in one or more embodiments,” “in certain embodiments,” “in one embodiment” or “in an embodiment” in various places throughout this specification are not necessarily referring to the same embodiment of the invention. Furthermore, the particular features, structures, materials, or characteristics may be combined in any suitable manner in one or more embodiments.

As used herein, the singular forms “a,” “an,” and “the” include plural references unless the context clearly indicates otherwise. Thus, for example, reference to “a polymer” includes a single polymer as well as a mixture of two or more polymers.

As used herein, the term “about” in connection with a measured quantity, refers to the normal variations in that measured quantity as expected by one of ordinary skill in the art in making the measurement and exercising a level of care commensurate with the objective of measurement and the precision of the measuring equipment. In certain embodiments, the term “about” includes the recited number ±10%, such that “about 10” would include from 9 to 11.

The term “at least about” in connection with a measured quantity refers to the normal variations in the measured quantity, as expected by one of ordinary skill in the art in making the measurement and exercising a level of care commensurate with the objective of measurement and precisions of the measuring equipment and any quantities higher than that. In certain embodiments, the term “at least about” includes the recited number minus 10% and any quantity that is higher such that “at least about 10” would include 9 and anything greater than 9. This term can also be expressed as “about 10 or more.” Similarly, the term “less than about” typically includes the recited number plus 10% and any quantity that is lower such that “less than about 10” would include 11 and anything less than 11. This term can also be expressed as “about 10 or less.”

The term “immediate release” refers to a dosage form (e.g., softgel capsule formulation) as disclosed herein that releases at least about 85 wt. %, at least about 90 wt. %, or at least about 95 wt. % of the active agent (or cosmetic agent), that is encapsulated within the shell composition, within about 30 minutes, or within about 45 minutes, or within about 60 minutes, as measured by in-vivo dissolution in a USP Apparatus II at about 50 RPM to about 250 RPM in 900 ml at 0.1N HCl (optionally with Pepsin) at 37° C.

The term “controlled release,” refers to a dosage form (e.g., softgel capsule formulation) as disclosed herein that releases the active agent (or cosmetic agent) that is encapsulated within the shell composition over a period of time, e.g., to provide a once daily or twice daily dosage form.

The term “softgel,” as used herein does not imply that gelatin is necessarily part of the shell composition (or film composition) and/or of the fill material (or fill composition). In certain embodiments, the shell composition (or film composition) may include gelatin, while in other embodiments, the shell composition may be free of gelatin. Similarly, in certain embodiments, the fill material (or fill composition) may include gelatin, while in other embodiments, the fill material may be free of gelatin. The term “softgel” may be used interchangeably with the term “softshell” throughout the description.

The term “shell composition” may be used interchangeably with the terms “film composition,” “shell.” and “film” throughout the description. These terms refer to the outer portion of the softgel composition (for instance, these terms refer to the shell of a softgel capsule which encapsulates a fill material).

The term “fill material” may be used interchangeably with the terms “fill composition.” and “fill” throughout the description. These terms refer to the inner portion of the softgel composition that is encapsulated by the shell composition (for instance, the inner portion of a softgel capsule).

The term “softgel composition” may be used interchangeably with the terms “softgel formulation,” and “dosage form” throughout the description. In certain embodiments, the term “softgel composition” or the term “softgel formulation” may be used interchangeably with the terms “softgel capsule composition” or the term “softgel capsule formulation,” respectively.

Unless otherwise indicated, all parts and percentages are by weight. Weight percent (wt. %), if not otherwise indicated, is based on an entire composition free of any volatiles, that is, based on dry solids content.

Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context.

The use of any and all examples, or exemplary language (e.g., “such as”) provided herein, is intended merely to illuminate certain materials and methods and does not pose a limitation on scope. No language in the specification should be construed as indicating any non-claimed element as essential to the practice of the disclosed materials and methods.

Although the disclosure herein is with reference to particular embodiments, it is to be understood that these embodiments are merely illustrative of the principles and applications of the invention. It will be apparent to those skilled in the art that various modifications and variations can be made to the compositions and methods without departing from the spirit and scope of the invention. Thus, it is intended that the invention include modifications and variations that am within the scope of the appended claims and their equivalents.

### Softgel Capsule Formulations

Disclosed herein is a softgel capsule shell composition comprising a non-animal derived gelling agent and a water soluble polymer. In certain embodiments, the shell composition completely dissolves in less than 30 minutes when subject to a dissolution with a USP Apparatus II with paddles at 50-250 RPM (e.g., 75 RPM) in 900 ml of 0.1N HCL and deionized water at 37 degrees C. In certain embodiments, the softgel capsule shell composition dissolves in less than 25 minutes, less than 20 minutes, less than 15 minutes, less than 10 minutes, or less than 5 minutes.

In certain embodiments of the softgel capsule shell composition, the non-animal derived gelling agent comprises carrageenan and starch. The weight ratio of carrageenan to starch in the softgel capsule shell composition may be, e.g., about 1:1 to about 1:10, about 1:1 to about 1:8, about 1:1 to about 1:5, or about 1:2.5 to about 1:4.5. In certain embodiments, the inclusion of a water soluble polymer in the softgel capsule shell composition contributes to the weight ratio of carrageenan to starch being less critical (e.g., with respect to dissolution, disintegration, elasticity, strength, and so on) than it would have otherwise been if the softgel capsule shell composition did not include a water soluble polymer as described herein.

Also disclosed in certain embodiments is a softgel capsule formulation comprising a fill material comprising an active agent, wherein the fill material is encapsulated by a shell composition as disclosed herein. The fill material can comprise, e.g., a hydrophilic material, a lipophilic, an amphiphilic material, or a combination thereof with an optional surfactant. The fill material can be a solution, suspension, semi solid, or solid and can further include an active agent (e.g., an active pharmaceutical ingredient or a nutraceutical), or a cosmetic agent.

The shell composition as disclosed herein may also contain at least one of a buffering agent, a plasticizer, and water. Softgel capsule formulations as described herein can be vegetarian and free of animal derived materials such as gelatin. In certain embodiments, the shell composition can comprise less than 10 wt. %, less than 5 wt. %, less than 1 wt. %, or 0 wt. % of an animal derived gelling agent.

In certain embodiments, the shell composition disclosed herein completely dissolves in less than 25 minutes, less than 20 minutes, less than 15 minutes, less than 10 minutes, or less than 5 minutes when subject to a dissolution with a USP Apparatus II with paddles at about 50 RPM to about 250 RPM (e.g., about 50 RPM, or at about 75 RPM, or at about 100 RPM, or at about 150 RPM, or at about 200 RPM, or at about 250 RPM) in 900 ml of 0.1N HCL and deionized water (optionally with Pepsin) at 37 degrees C.

In certain embodiments, the shell composition disclosed herein completely dissolves in any of about 5 minute, about 8 minutes, about 10 minutes, about 12 minutes, or about 15 minutes to any of about 20 minutes, about 23 minutes, about 25 minutes, about 28 minutes, about 30 minutes, or about 35 minutes when subject to a dissolution with a USP Apparatus II with paddles at about 50 RPM to about 250 RPM (e.g., about 50 RPM, or at about 75 RPM, or at about 100 RPM, or at about 150 RPM, or at about 200 RPM, or at about 250 RPM) in 900 ml of 0.1N HCL and deionized water (optionally with Pepsin) at 37 degrees C.

In certain embodiments, the shell composition disclosed herein begins dissolving in less than 15 minutes, less than 10 minutes, less than 9 minutes, less than 8 minutes, or less than 7 minutes when subject to a dissolution with a USP Apparatus II with paddles at about 50 RPM to about 250 RPM (e.g., about 50 RPM, or at about 75 RPM, or at about 100 RPM, or at about 150 RPM, or at about 200 RPM, or at about 250 RPM) in 900 ml of 0.1N HCL and deionized water (optionally with Pepsin) at 37 degrees C.

In certain embodiments, the shell composition disclosed herein begins dissolving in any of about 1 minute, about 2 minutes, about 3 minutes, about 4 minutes, or about 5 minutes to any of about 7 minutes, about 8 minutes, about 9 minutes, about 10 minutes, or about 15 minutes when subject to a dissolution with a USP Apparatus II with paddles at about 50 RPM to about 250 RPM (e.g., about 50 RPM, or at about 75 RPM, or at about 100 RPM, or at about 150 RPM, or at about 200 RPM, or at about 250 RPM) in 900 ml of 0.1N HCL and deionized water (optionally with Pepsin) at 37 degrees C.

In certain embodiments, softgel compositions (e.g., softgel capsules) that include a fill material encapsulated with the shell composition disclosed herein begin rupturing in less than 25 minutes, less than 20 minutes, less than 15 minutes, less than 10 minutes, or less than 5 minutes when subject to a disintegration with a USP Disintegration Apparatus with paddles at about 50 RPM to about 250 RPM (e.g., about 50 RPM, or at about 75 RPM, or at about 100 RPM, or at about 150 RPM, or at about 200 RPM, or at about 250 RPM) in 900 ml of 0.1N HCL and deionized water (optionally with Pepsin) at 37 degrees C.

In certain embodiments, softgel compositions (e.g., softgel capsules) that include a fill material encapsulated with the shell composition disclosed herein begin rupturing in any of about 1 minute, about 2 minutes, about 3 minutes, about 4 minutes, about 5 minutes, or about 7 minutes to any of about 8 minutes, about 9 minutes, about 10 minutes, about 15 minutes, or about 20 minutes when subject to a disintegration with a USP Disintegration Apparatus with paddles at about 50 RPM to about 250 RPM (e.g., about 50 RPM, or at about 75 RPM, or at about 100 RPM, or at about 150 RPM, or at about 200 RPM, or at about 250 RPM) in 900 ml of 0.1N HCL, and deionized water (optionally with Pepsin) at 37 degrees C.

In certain embodiments, softgel compositions (e.g., softgel capsules) that include a fill material encapsulated with the shell composition disclosed herein completely dissolves in less than 45 minutes, less than 40 minutes, less than 35 minutes, less than 30 minutes, or less than 25 minutes when subject to a disintegration with a USP Disintegration Apparatus with paddles at about 50 RPM to about 250 RPM (e.g., about 50 RPM, or at about 75 RPM, or at about 100 RPM, or at about 150 RPM, or at about 200 RPM, or at about 250 RPM) in 900 ml of 0.1N HCL and deionized water (optionally with Pepsin) at 37 degrees C.

In certain embodiments, softgel compositions (e.g., softgel capsules) that include a fill material encapsulated with the shell composition disclosed herein completely dissolves in any of about 15 minutes, about 18 minutes, about 20 minutes, about 22 minutes, or about 25 minutes to any of about 30 minutes, about 35 minutes, about 40 minutes, or about 45 minutes when subject to a disintegration with a USP Disintegration Apparatus with paddles at about 50 RPM to about 250 RPM (e.g., about 50 RPM, or at about 75 RPM, or at about 100 RPM, or at about 150 RPM, or at about 200 RPM, or at about 250 RPM) in 900 ml of 0.1N HCL and deionized water (optionally with Pepsin) at 37 degrees C.

It should be understood that the dissolution and/or disintegration times of the shell composition may be independent from the dissolution and/or disintegration times of the fill material. The release profile of the active agent from a dosage form may depend on the shell composition and on the fill composition. The dissolution and/or disintegration time of the shell composition may be indicative, at least in part, of the release profile of the active agent from a dosage form. Dosage forms, according to embodiments described herein, may be formulated as immediate release dosage forms (e.g., by formulating an immediate release shell composition with an immediate release fill material) and as controlled release dosage forms (e.g., by formulating an immediate release shell composition with a controlled release fill material).

According to certain embodiments, the water soluble polymer may comprise, e.g., polyvinyl alcohol (PVA), pullulan gum, polylactic acid, polyvinyl alcohol-polyethylene glycol graft co-polymer (PVA-PEG copolymer), high molecular weight polyethylene glycol, povidone, a surfactant (e.g., sodium lauryl sulfate), or a combination thereof. In one embodiment, the water soluble polymer is PVA. In one embodiment, the water soluble polymer is PVA-PEG copolymer. In one embodiment, the water soluble polymer is pullulan gum. High molecular weight polyethylene glycol may be polyethylene glycol having a number average molecular weight ranging from about 600 Da to about 2,000.000 Da and any number average molecular weight therein (e.g., PEG 600, PEG 800, PEG 1000, PEG 1500, PEG 3350, PEG 4000, PEG 6000, PEG 8000, and so on). In certain embodiments, the water soluble polymer is in the shell composition in an amount of, e.g., about 0.5 wt. % to about 10 wt. %, about 0.5 wt. % to about 12 wt. %, about 1 wt. % to about 15 wt. %, about 1 wt. % to about 20 wt. %, about 2 wt. % to about 22 wt. %, about 2 wt. % to about 7 wt. %, about 0.5 wt. % to about 8 wt. %, about 3 wt. % to about 9 wt. %, about 2.5 wt. % to about 30 wt. %, about 10 wt. % to about 50 wt. %, or about 20 wt. % to about 40 wt. %, or about 15 wt. % to about 30 wt. %, or about 15 wt. %, or about 18 wt. %, or about 20 wt. %, or about 22 wt. %, or about 25 wt. %, or about 28 wt. %, or about 30 wt. %, or any sub-range or single concentration value therein, with all wt. % being based on the total weight of the shell composition.

According to embodiments, the non-animal gelling agent may include, e.g., carrageenan, starch, pregelatinized starch, xanthan gum, agar, pectin, alginate, sugar, sugar derived alcohol, monosaccharides, disaccharides, oligosaccharides, a cellulose derivative, a cellulosic polymer, hydroxyethylcellulose, hydroxypropylcellulose, hydroxypropylmethylcellulose, carboxymethylcellulose, microcrystalline cellulose, attapulgite, bentonite, dextrin, alginate, kaolin, lecithin, magnesium aluminum silicate, carbomer, carbopol, silicon dioxide, curdlan, furcelleran, albumin (e.g., egg or lacto derived), soy protein, chitosan, guaic gum, tamarind seed polysaccharide, glucomannan, chitin, pluran, cyclodextrin, or a combination thereof.

The carrageenan can be at least one of iota carrageenan, kappa carrageenan and lambda carrageenan.

The starch can be modified starch or native starch, sweet potato starch, potato starch, corn starch, tapioca starch, pea starch, hydroxy propylated starch, hydroxyalkylated starch, acid-treated starch, dextrin, high amylose non-modified corn starch, modified waxy maize starch, non-granular starch, modified high amylose corn starch, pregelatinized rice flour and a combination thereof. As used herein and in the claims, the term “modified starch” includes such starches as hydroxypropylated starches, acid thinned starches and the like. In general, modified starches are products prepared by chemical treatment of starches, for example, acid treatment starches, enzyme treatment starches, oxidized starches, cross-bonding starches, and other starch derivatives. It is preferred that the modified starches be derivatized wherein side chains are modified with hydrophilic or hydrophobic groups to thereby form a more complicated structure with a strong interaction between side chains.

In certain embodiments, the non-animal gelling agent is in the shell composition in an amount, e.g., of about 2 wt. % to about 20 wt. %, about 2 wt. % to about 15 wt. %, about 2 wt. % to about 40 wt. %, about 10 wt. % to about 80 wt. %, or about 15 wt. % to about 75 wt. %, or about 20 wt. % to about 70 wt. %, or about 25 wt. % to about 60 wt. %, or about 25 wt. % to about 45 wt. %, or about 20 wt. % to about 35 wt. %, or about 30 wt. % to about 40 wt. %, or about 32 wt. %, or about 35 wt. %, or about 38 wt. %, or any sub-range or single concentration value therein, with all wt. % being based on the total weight of the shell composition. In one embodiment, the non-animal gelling agent includes carrageenan and does not include starch (or modified starch). In one embodiment, the softgel shell composition is substantially free or free of starch (or modified starch).

In certain embodiments the weight ratio of the water soluble polymer (e.g., PVA, PVA-PEG copolymer, pullulan, or combination thereof) to the non-animal gelling agent (e.g., carrageenan, starch, or combination thereof), may range from about 20:1 to about 1:20, from about 15:1 to about 1:15, from about 10:1 to about 1:10, from about 8:1 to about 1:8, from about 5:1 to about 1:5, from about 3:1 to about 1:3, or from about 2:1 to about 1:2, or about 1:1. Without being construed as limiting, it is believed that the weight ratio of the water soluble polymer to the non-animal gelling agent contributes to the dissolution time of the softgel shell compositions described herein.

According to embodiments, the buffer agent includes at least one of dibasic sodium phosphate, monobasic sodium phosphate, sodium bicarbonate, sodium citrate, disodium phosphate, calcium phosphate, dibasic calcium phosphate, tribasic calcium phosphate, monobasic potassium phosphate, and dibasic potassium phosphate. In embodiments, the buffer agent comprises dibasic sodium phosphate. In embodiments, the buffering agent is in the shell composition in an amount of about 0.01 wt. % to about 5 wt. %, or about 0.05 wt. % to about 4 wt. %, or about 0.1 wt. % to about 3 wt. %, or about 0.5 wt. % to about 3 wt. %, or about 1.0 wt. %, or any sub-range or single concentration value therein, with all wt. % being based on the total weight of the shell composition.

According to various embodiments, the plasticizer may comprise, e.g., glycerin, sorbitol, sorbitol and sorbitan solution, triacetin, polysorbate, propylene glycol, sodium lauryl sulfate (SLS), sugar alcohols (e.g., maltitol), or combinations thereof. In certain embodiments, the plasticizer is glycerin. In one embodiment, the plasticizer is sorbitol. In one embodiment, the plasticizer is SLS. In embodiments, the plasticizer is in the shell composition in an amount of about 0.5 wt. % to about 40.0 wt. %, 10 wt. % to about 30.0 wt. %, or about 12 wt. % to about 28 wt. %, or about 15 wt. % to about 30 wt. %, or about 18 wt. % to about 23 wt. %, or about 18 wt. %, or about 20 wt. %, or about 22 wt. %, or about 25 wt. %, or about 28 wt. %, or about 30 wt. %, or any sub-range or single concentration value therein, with all wt. % being based on the total weight of the shell composition.

In certain embodiments, the softgel capsule shell composition contains water. The water may be present in the shell composition in an amount of about 30 wt. % to about 60 wt./, or about 35 wt. % to about 55 wt. %, or about 40 wt. % to about 50 wt. %, or about 42 wt. %, or about 43 wt. %, or about 44 wt. %, or about 45 wt. %, or about 45.5 wt. %, or about 46 wt. %, or about 47 wt. %, or about 48 wt. %, or any sub-range or single concentration value therein, with all wt. % being based on the total weight of the shell composition. According to certain embodiments, a weight ratio of the water to the non-animal gelling agent in the shell composition is about 1:5 to about 5:1, or about 1:4 to about 4:1, or about 1:3 to about 3:1, or about 1:1, or about 2:1, or about 3:1, or about 4:1, or about 5:1.

In certain embodiments, the softgel shell compositions described herein have a film strength, as measured by a texture analyzer in accordance with the details described in the examples, of greater than about 5.0 kg, greater than about 5.2 kg, or greater than about 5.4 kg. In certain embodiments, the softgel shell compositions described herein have a film strength ranging from any of about 3.5 kg, about 4.0 kg, about 4.5 kg, about 5.0 kg, or about 5.5 kg to any of about 6.0 kg, about 6.5 kg, about 7.0 kg, about 7.5 kg, about 8.0 kg, about 8.5 kg, about 9.0 kg, about 9.5 kg, about 10.0 kg, about 10.5 kg, about 11.0 kg, about 11.5 kg, or about 12.0 kg, or any single value or sub-range therein. In certain embodiments, the softgel shell compositions described herein have a film strength ranging from about 3.5 kg to about 12.0 kg, from about 5.0 kg to about 12.0 kg, from about 5.2 kg to about 10.0 kg, or from about 5.0 kg to about 7.0 kg.

In certain embodiments, the softgel shell compositions described herein have an elasticity, as measured by a texture analyzer in accordance with the details described in the examples, of greater than about 7.7 mm, greater than about 8.0 mm, or greater than about 8.2 mm. In certain embodiments, the softgel shell compositions described herein have an elasticity ranging from any of about 6 mm, about 6.5 mm, about 7.0 mm, about 7.5 mm, about 8.0 mm, or about 8.5 mm to any of about 9.0 mm, about 9.5 mm, about 10.0 mm, about 10.5 mm, about 11.0 mm, about 11.5 mm, or about 12.0 mm, or any single value or sub-range therein. In certain embodiments, the softgel shell compositions described herein have an elasticity of about 6.0 mm to about 12.0 mm, about 7.0 mm to about 10.0 mm, about 8.0 mm to about 12.0 mm, or about 8.0 mm to about 10.0 mm.

In certain embodiments, the softgel shell compositions described herein have sufficient elasticity to allow for the formation of capsules, while also being strong enough to survive manipulation in the encapsulation machine (e.g., rotary die), and provide good sealing properties at temperatures below the melting point of the softgel shell composition, all without compromising the dissolution or disintegration profiles of the softgel shell compositions and of the capsule.

The softgel capsule formulations as disclosed herein may further include a fill material. The fill material may contain at least one of rapeseed oil, medium chain triglyceride oil, polyethylene glycol, and combinations thereof. Lipophilic and/or hydrophilic and/or alcohol fill materials could also be encapsulated with the softgel capsule fill materials described herein.

### Active Agents

Any pharmaceutically active ingredient may be used for purposes of the present invention, including both those that are water-soluble and those that are poorly soluble in water. Suitable pharmaceutically active ingredients include, without limitation, analgesics and anti-inflammatory agents, antacids, anthelmintic, anti-arrhythmic agents, anti-bacterial agents, anti-coagulants, anti-depressants, anti-diabetics, anti-diarrheal, anti-epileptics, anti-fungal agents, anti-gout agents, anti-hypertensive agents, anti-malarial, anti-migraine agents, anti-muscarinic agents, anti-neoplastic agents and immunosuppressant agents, anti-protozoal agents, anti-rheumatics, anti-thyroid agents, antivirals, anxiolytics, sedatives, hypnotics and neuroleptics, beta-blockers, cardiac inotropic agents, corticosteroids, cough suppressants, cytotoxic agents, decongestants, diuretics, enzymes, anti-parkinsonian agents, gastro-intestinal agents, histamine receptor antagonists, lipid regulating agents, local anesthetics, neuromuscular agents, nitrates and anti-angina agents, nutritional agents, opioid analgesics, oral vaccines, proteins, peptides and recombinant drugs, sex hormones and contraceptives, spermicides, stimulants, and combinations thereof.

In some embodiments, the active pharmaceutical ingredient may be selected, without limitations, from the group of dabigatran, dronedarone, ticagrelor, iloperidone, ivacaftor, midostaurine, asimadoline, beclomethasone, apremilast, sapacitabine, linsitinib, abiraterone, vitamin D analogs (e.g., calcifediol, calcitriol, paricalcitol, doxercalciferol), COX-2 inhibitors (e.g., celecoxib, valdecoxib, rofecoxib), tacrolimus, testosterone, lubiprostone, pharmaceutically acceptable salts thereof, and combinations thereof.

In some embodiments, the lipids in the dosage form may be selected, without limitations, from the group of almond oil, argan oil, avocado oil, borage seed oil, canola oil, cashew oil, castor oil, hydrogenated castor oil, cocoa butter, coconut oil, colza oil, corn oil, cottonseed oil, grape seed oil, hazelnut oil, hemp oil, hydroxylated lecithin, lecithin, linseed oil, macadamia oil, mango butter, manila oil, mongo go nut oil, olive oil, palm kernel oil, palm oil, peanut oil, pecan oil, perilla oil, pine nut oil, pistachio oil, poppy seed oil, pumpkin seed oil, rice bran oil, safflower oil, sesame oil, shea butter, soybean oil, sunflower oil, hydrogenated vegetable oil, walnut oil, and watermelon seed oil. Other oil and fats may include, but not be limited to, fish oil (omega-3), krill oil, animal or vegetable fats, e.g., in their hydrogenated form, free fatty acids and mono-, di-, and tri-glycerides with C8-, C10-, C12-, C14-, C16-, C18-, C20- and C22-fatty acids, and combinations thereof.

According to certain embodiments, active agents may include lipid-lowering agents including, but not limited to, statins (e.g., lovastatin, simvastatin, pravastatin, fluvastatin, atorvastatin, rosuvastatin, and pitavastatin), fibrates (e.g., clofibrate, ciprofibrate, bezafibrate, fenofibrate, and gemfibrozil), niacin, bile acid sequestrants, ezetimibe, lomitapide, phytosterols, and the pharmaceutically acceptable salts, hydrates, solvates and prodrugs thereof, mixtures of any of the foregoing, and the like.

Suitable nutraceutical active agents may include, but are not limited to, 5-hydroxytryptophan, acetyl L-carnitine, alpha lipoic acid, alpha-ketoglutarates, bee products, betaine hydrochloride, bovine cartilage, caffeine, cetyl myristoleate, charcoal, chitosan, choline, chondroitin sulfate, coenzyme Q10, collagen, colostrum, creatine, cyanocobalamin (Vitamin 812), dimethylaminoethanol, fumaric acid, germanium sequioxide, glandular products, glucosamine HCl, glucosamine sulfate, hydroxyl methyl butyrate, immunoglobulin, lactic acid, L-Carnitine, liver products, malic acid, maltose-anhydrous, mannose (d-mannose), methyl sulfonyl methane, phytosterols, picolinic acid, pyruvate, red yeast extract, S-adenosylmethionine, selenium yeast, shark cartilage, theobromine, vanadyl sulfate, and yeast.

Suitable nutritional supplement active agents may include vitamins, minerals, fiber, fatty acids, amino acids, herbal supplements or a combination thereof.

Suitable vitamin active agents may include, but are not limited to, the following: ascorbic acid (Vitamin C), B vitamins, biotin, fat soluble vitamins, folic acid, hydoxycitric acid, inositol, mineral ascorbates, mixed tocopherols, niacin (Vitamin B3), orotic acid, para-aminobenzoic acid, panthothenates, panthothenic acid (Vitamin B5), pyridoxine hydrochloride (Vitamin B6), riboflavin (Vitamin B2), synthetic vitamins, thiamine (Vitamin B1), tocotrienols, vitamin A, vitamin D, vitamin E, vitamin F, vitamin K, vitamin oils and oil soluble vitamins.

Suitable herbal supplement active agents may include, but are not limited to, the following: arnica, bilbery, black cohosh, cat's claw, chamomile, echinacea, evening primrose oil, fenugreek, flaxseed, feverfew, garlic, ginger root, ginko biloba, ginseng, goldenrod, hawthorn, kava-kava, licorice, milk thistle, psyllium, rauowolfia, senna, soybean, St. John's wort, saw palmetto, turmeric, valerian.

Minerals active agents may include, but are not limited to, the following: boron, calcium, chelated minerals, chloride, chromium, coated minerals, cobalt, copper, dolomite, iodine, iron, magnesium, manganese, mineral premixes, mineral products, molybdenum, phosphorus, potassium, selenium, sodium, vanadium, malic acid, pyruvate, zinc and other minerals.

Examples of other possible active agents include, but are not limited to, antihistamines (e.g., ranitidine, dimenhydrinate, diphenhydramine, chlorpheniramine and dexchlorpheniramine maleate), non-steroidal anti-inflammatory agents (e.g., aspirin, celecoxib, Cox-2 inhibitors, diclofenac, benoxaprofen, flurbiprofen, fenoprofen, flubufen, indoprofen, piroprofen, carprofen, oxaprozin, pramoprofen, muroprofen, trioxaprofen, suprofen, aminoprofen, fluprofen, bucloxic acid, indomcthacin, sulindac, zomcpirac, tiopinac, zidometacin, acemetacin, fentiazac, clidanac, oxpinac, meclofenamic acid, flufenamic acid, niflumic acid, tolfenamic acid, diflurisal, flufenisal, piroxicam, sudoxicam, isoxicam, aceclofenac, aloxiprin, azapropazone, benorilate, bromfenac, carprofen, choline magnesium salicylate, diflunisal, etodolac, etoricoxib, faislamine, fenbufen, fenoprofen, flurbiprofen, ibuprofen, indometacin, ketoprofen, ketorolac, lomoxicam, loxoprofen, meloxicam, mefenamic acid, metamizole, methyl salicylate, magnesium salicylate, nabumetone, naproxen, nimesulide, oxyphenbutazone, parecoxib, phenylbutazone, salicyl salicylate, sulindac, sulfinpyrazone, tenoxicam, tiaprofenic acid, tolmetin, pharmaceutically acceptable salts thereof and mixtures thereof, acetaminophen, anti-emetics (e.g., metoclopramide, methylnaltrexone), anti-epileptics (e.g., phenyloin, meprobmate and nitrazepam), vasodilators (e.g., nifedipine, papaverine, diltiazem and nicardipine), anti-tussive agents and expectorants (e.g. codeine phosphate), anti-asthmatics (e.g. theophylline), antacids, anti-spasmodics (e.g. atropine, scopolamine), antidiabetics (e.g., insulin), diuretics (e.g., ethacrynic acid, bendrofluthiazide), anti-hypotensives (e.g., propranolol, clonidine), antihypertensives (e.g., clonidine, methyldopa), bronchodilatiors (e.g., albuterol), steroids (e.g., hydrocortisone, triamcinolone, prednisone), antibiotics (e.g., tetracycline), antihemorrhoidals, hypnotics, psychotropics, antidiarrheals, mucolytics, sedatives, decongestants (e.g. pseudoephedrine), laxatives, vitamins, stimulants (including appetite suppressants such as phenylpropanolamine) and cannabinoids, as well as pharmaceutically acceptable salts, hydrates, solvates, and prodrugs thereof.

The active agent that may also be a benzodiazepine, barbiturate, stimulants, or mixtures thereof. The term “benzodiazepines” refers to a benzodiazepine and drugs that an derivatives of a benzodiazepine that are able to depress the central nervous system.

Benzodiazepines include, but are not limited to, alprazolam, bromazepam, chlordiazepoxide, clorazepate, diazepam, estazolam, flurazepam, halazepam, ketazolam, lorazepam, nitrazepam, oxazepam, prazepam, quazepam, temazepam, triazolam, methylphenidate as well as pharmaceutically acceptable salts, hydrates, solvates, prodrugs and mixtures thereof. Benzodiazepine antagonists that can be used as active agent include, but are not limited to, flumazenil as well as pharmaceutically acceptable salts, hydrates, solvates and mixtures thereof.

The term “barbiturates” refers to sedative-hypnotic drugs derived from barbituric acid (2, 4, 6,-trioxohexahydropyrimidine). Barbiturates include, but are not limited to, amobarbital, aprobarbotal, butabarbital, butalbital, methohexital, mephobarbital, metharbital, pentobarbital, phenobarbital, secobarbital as well as pharmaceutically acceptable salts, hydrates, solvates, prodrugs, and mixtures thereof. Barbiturate antagonists that can be used as active agent include, but are not limited to, amphetamines as well as pharmaceutically acceptable salts, hydrates, solvates and mixtures thereof.

The term “stimulants” includes, but is not limited to, amphetamines such as dextroamphetamine resin complex, dextroamphetamine, methamphetamine, methylphenidate, as well as pharmaceutically acceptable salts, hydrates, and solvates and mixtures thereof. Stimulant antagonists that can be used as active agent include, but are not limited to, benzodiazepines, as well as pharmaceutically acceptable salts, hydrates, solvates and mixtures thereof.

The softgel capsule formulations according to the disclosure include various active agents and their pharmaceutically acceptable salts thereof. Pharmaceutically acceptable salts include, but are not limited to, inorganic acid salts such as hydrochloride, hydrobromide, sulfate, phosphate and the like; organic acid salts such as formate, acetate, trifluoroacetate, maleate, tartrate and the like; sulfonates such as methanesulfonate, benzenesulfonate, p-toluenesulfonate, and the like; amino acid salts such as arginate, asparginate, glutamate and the like, and metal salts such as sodium salt, potassium salt, cesium salt and the like; alkaline earth metals such as calcium salt, magnesium salt and the like; organic amine salts such as triethylamine salt, pyridine salt, picoline salt, ethanolamine salt, triethanolamine salt, dicyclohexylamine salt, N,N′-dibenzylethylenediamine salt and the like.

### Methods of Preparing the Softgel Capsule Formulation

Disclosed herein are methods of preparing a softgel shell composition and a softgel capsule formulation as disclosed herein. The methods include combining a non-animal gelling agent, a water soluble polymer, and optionally at least one of a buffering agent, a plasticizer or water to form a combination. The method further includes heating the combination to form a molten mass. In embodiments, the molten mass is a uniform molten mass. The method may further include extruding the molten mass to form ribbons. Additionally, the method may include casting the ribbons on drums and forming soft capsule shells using a rotary die encapsulation apparatus. In embodiments, the ribbons may have a thickness of e.g., about 0.001 in to about 0.100 in, about 0.001 in to about 0.070 in, about 0.001 in to about 0.050 in, or about 0.005 in to about 0.030 in, or about 0.010 in to about 0.025 in, or about 0.015 in to about 0.021 in, or about 0.017 in, or about 0.018 in, or about 0.019 in, or about 0.020 in, or about 0.021 in, or about 0.022 in. In certain embodiments, a netting can be formed from the combination. The netting can be subsequently melted and reused to form ribbons.

In one embodiment, the method may further include mixing the plasticizer with the water, if both are present, to form a plasticizer solution. The method may further include mixing the synthetic polymer, non-animal (natural) gelling agent, and buffering agent (if present) with the plasticizer solution. In the same embodiment or in alternative embodiment, the combining may further include mixing the plasticizer with the non-animal gelling agent to form a solution, mixing the solution with water to form a plasticizer solution, and mixing the water soluble polymer and buffering agent with the plasticizer solution (which include the non-animal gelling agent therein). The combining may comprise introducing each of the water soluble polymer, non-animal gelling agent, buffering agent, plasticizer and water into a low shear mixer. In embodiments, the combining can be for about 1 min to about 3 hours, or about 5 min to about 2.5 hours, or about 15 min to about 2.0 hours, or about 20 min to about 1.5 hours, or about 30 min to 1.0 hour, or about 5 min to about 30 min. The combining can be at a temperature of about 45° C. to about 90° C., or about 50° C. to about 85° C., or about 55° C. to about 80° C., or about 60° C. to about 70° C., or about 55° C., or about 60° C., or about 65° C. The combining can further include increasing the temperature to about 95° C. to about 125° C., or about 100° C. to about 120° C., or about 105° C. to about 115° C., or about 95° C., or about 96° C., or about 97° C., or about 98° C. or about 99° C., or about 100° C.

According to certain embodiments, the method may further include transferring the combination to a receiving tank. The combination or material in the receiving tank may be transferred to a heated vessel to heat the material therein. In certain embodiments, the heated vessel may heat the combination to a temperature of about 80° C. to about 115° C., or about 85° C. to about 100° C., or about 88° C. to about 95° C. According to certain embodiments, the method may include injecting a coloring agent into the combination.

The method may also further include transferring the combination to an encapsulation apparatus. In certain embodiments, the method may include encapsulating a fill material within a softgel capsule fill material, formed from any of the above-described combinations, to form a plurality softgel capsule dosage forms. The method may also further include drying the plurality of softgel capsule dosage forms in a tumble dryer. Certain embodiments further include packaging the plurality of softgel capsule dosage forms.

### Methods of Treatment

Disclosed herein are methods of treating, preventing, minimizing or ameliorating a condition, treatable by the active agents included in the fill material of the softgel capsules described herein, in a subject in need thereof. The methods include administering any of the softgel capsules described herein to the subject in need thereof, whereby the condition is treated, prevented, minimized, ameliorated, or reduced upon administration of the softgel capsules.

The terms “treatment of” and “treating” include the administration of an active agent(s) with the intent to lessen the severity of a condition.

The terms “prevention of” and “preventing” include the avoidance of the onset of a condition by a prophylactic administration of the active agent.

The term “condition” or “conditions” may refer to those medical conditions that may be treated, prevented, minimized, or ameliorated by administration of an effective amount of any of the active agents or cosmetic agents described herein. In certain embodiments, the term “condition” or “conditions” may refer to pain.

In certain embodiments, the softgel capsules described herein (e.g., those that include a non-animal derived gelling agent, water soluble polymer, and at least one of buffering agent, plasticizer, and water) contribute to a faster Tₘₐₓ as compared to softgel capsules that do not include one of the components recited hereinabove (such as the water soluble polymer).

Tₘₐₓ refers to the time for the plasma concentration of an active agent to reach a Cₘₐₓ.

Cₘₐₓ refers to the maximum plasma concentration of an active agent.

As used herein, the terms “effective amount” refer to the amount and/or the rate of an active agent or of a cosmetic agent needed to produce a desired therapeutic or cosmetic result.

The term “subject” refers to a human or animal who has demonstrated a manifestation (clinical or otherwise) of a condition suggesting the need for treatment with any of the active agents described herein.

### EXAMPLES

Specific embodiments of the disclosure will now be demonstrated by reference to the following examples. It should be understood that these examples are disclosed solely by way of illustrating the disclosure and should not be taken in any way to limit the scope of the present disclosure.

### Example 1

Comparison of softgel capsule formulation to non-animal gelling agent capsule shell composition without a water soluble polymer (control) and with a water soluble polymer. The dissolution is tested with a USP Apparatus II with paddles at 75 RPM in 900 ml of 0.1N HCL and deionized water at 37 degrees C. The test shell composition is a sample that is 1.0 inch (25.4 mm) in diameter and 0.7 mm and 1.4 mm thick.

TABLE 1

| | Formula | | | | | | | | | |
|---|---|---|---|---|---|---|---|---|---|---|
| | Control | | Exp 13 - PVA-PEG | | Exp 41 - PVA w. | | Exp 14 - PVA-PEG | | | |
| | (Current) | | copolymer | | SLS | | copolymer | | Exp 24 - PVA | |
| Ingredient | Wt. % | Ratio* | Wt. % | Ratio* | Wt. % | Ratio* | Wt. % | Ratio* | Wt. % | Ratio* |
| Modified | 20-32 | 3.0-4.5 | 16-25 | 2.5-4 | 16-25 | 2.5-4 | 16-25 | 2.5-4 | 16-25 | 2.5-4 |
| Starch | | | | | | | | | | |
| Carrageenan | 4-12 | 1.0 | 4-10 | 1.0 | 3-9 | 1.0 | 2.5-8 | 1.0 | 3.5-11 | 1.0 |
| Buffer Agent | 0.8-1.2 | 0.3-1.1 | 0.2-1.5 | 0.04-0.6 | 0.2-1.5 | 0.04-0.6 | 0.2-1.5 | 0.04-0.6 | 0.2-1.5 | 0.04-0.6 |
| (Sodium | | | | | | | | | | |
| Phosphate | | | | | | | | | | |
| dibasic) | | | | | | | | | | |
| Plasticizer | 10-30 | 1.8-4.0 | 15-30 | 1.5-7.2 | 15-30 | 1.5-7.2 | 15-30 | 1.5-7.2 | 15-30 | 1.5-7.2 |
| (Polysorb) | | | | | | | | | | |
| Water | 0 | 0 | 0.5-10 | 0.05-1 | — | — | 0.4-8 | 0.06-1.2 | — | — |
| soluble | | | | | | | | | | |
| polymer - | | | | | | | | | | |
| Kollicoat IR | | | | | | | | | | |
| (polyvinyl | | | | | | | | | | |
| alcohol- | | | | | | | | | | |
| polyethylene | | | | | | | | | | |
| glycol graft | | | | | | | | | | |
| copolymer) | | | | | | | | | | |
| Water | 0 | 0 | — | — | 2.0-7.0 | 0.3-1.6 | — | — | 3.0-9.0 | 0.4-1.8 |
| soluble | | | | | | | | | | |
| polymer - | | | | | | | | | | |
| Polyvinyl | | | | | | | | | | |
| Alcohol | | | | | | | | | | |
| (PVA) | | | | | | | | | | |
| Kolliphor | 0 | 0 | — | — | 0.5-3.0 | 0.1-0.6 | — | — | — | — |
| SLS (sodium | | | | | | | | | | |
| lauryl | | | | | | | | | | |
| sulfate)- | | | | | | | | | | |
| plasticizer | | | | | | | | | | |
| Water | 35-55 | — | 35-55 | — | 35-55 | — | 35-55 | — | 35-55 | — |
| Complete | Water | 0.1N | Water | 0.1N | Water | 0.1N | Water | 0.1N | Water | 0.1N |
| film | | HCl | | HCl | | HCl | | HCl | | HCl |
| dissolution | | | | | | | | | | |
| (min) | | | | | | | | | | |
| Sample 1 | 63.25 | — | 12.56 | 19.27 | 10.46 | 18.2 | 15.18 | 23.02 | 12.56 | 19.27 |
| Sample 2 | 71.05 | — | 18.22 | 19.48 | 9.56 | 17.39 | 11.57 | 21.39 | 18.22 | 19.48 |
| Sample 3 | — | — | 12.08 | 22.0 | 10.05 | 17.26 | 9.48 | 24.49 | 12.08 | 22.0 |
| Sample 4 | — | — | 12.31 | 24.55 | 10.13 | 22.32 | 15.31 | 24.26 | 12.31 | 24.55 |
| Sample 5 | — | — | 16.47 | 22.11 | 11.1 | 22.09 | 15.02 | 21.00 | 16.47 | 22.11 |
| Sample 6 | — | — | 15.19 | 21.32 | 10.22 | 20.3 | 13.37 | 20.48 | 15.19 | 21.32 |
| Average | — | — | 14.47 | 21.46 | 10.25 | 19.59 | 13.32 | 22.44 | 14.47 | 21.46 |
| (minutes) | | | | | | | | | | |

*Weight ratio with respect to Carrageenan

As can be seen in Table 1, the control non-animal gelling agent capsule shell composition without a water soluble polymer had a dissolution time in water of 63.25 minutes and 71.05 minutes. In comparison, each non-animal gelling agent capsule shell composition with a water soluble polymer (whether PVA by itself, PVA with SLS, or PVA-PEG copolymer), all had a much faster dissolution in water (less than 20 minutes) and in 0.1N HCl (less than 25 minutes).

Interestingly, faster dissolution was observed in non-animal gelling agent capsule shell composition that included PVA with SLS than any of the other water soluble polymers. The shell composition that included PVA without SLS exhibited a similar dissolution to shell composition that include PVA-PEG copolymer as the water soluble polymer.

Without being construed as limiting, it is believed that the type of the water soluble polymer as well as the weight ratio of the carrageenan to the particular water soluble polymer may affect the dissolution time of the shell composition.

### Example 2

The solubility of various softgel shell compositions was evaluated by incorporating natural or synthetic water soluble polymers as shown in Table 2 below.

TABLE 2
Softgel Shell compositions

| | F-1 | F-2 | F-3 | F-4 | F-5 | F-6 |
|---|---|---|---|---|---|---|
| Ingredient | Wt. % | Wt. % | Wt. % | Wt. % | Wt. % | Wt. % |
| Carrageenan | 2-20 | 2-15 | 2-15 | 2-15 | 2-15 | 2-15 |
| Plasticizer (e.g., sorbitol) | 8-45 | 8-45 | 8-45 | 8-45 | 8-45 | 8-45 |
| Sodium Phosphate Dibasic | 0.2-4 | 0.2-4 | 0.2-4 | 0.2-4 | 0.2-4 | 0.2-4 |
| (Buffer Agent) | | | | | | |
| Polyvinyl alcohol (PVA) | — | 0.5-12 | — | — | 1-10 | 1-10 |
| (water soluble polymer) | | | | | | |
| Pullulan (water soluble | — | — | 1-15 | — | 1-12 | 1-12 |
| polymer) | | | | | | |
| Polyvinyl alcohol- | — | — | — | 1-20 | — | 0.5-8 |
| polyethylene glycol | | | | | | |
| copolymer (PVA-PEG | | | | | | |
| copolymer) (water soluble | | | | | | |
| polymer) | | | | | | |

Softgel shell compositions per above formulations (F-1 trough F-6) were prepared. Formulations F-1 through F-4 were cast into films. Shell compositions F-1 through F-4 were allowed to dry to moisture of 6 wt. %-15 wt. %, based on total weight of the shell composition. The dried shell compositions were evaluated for film strength and elasticity using a Texture Analyzer. Table 3 summarizes the strength and elasticity of shell compositions prepared using various water soluble polymers. The data is generated using dry shell compositions containing 6 wt.-15 wt. % moisture, based on total weight of the softgel shell composition.

The Texture Analyzer test conditions for measuring strength and elasticity were as follows: the softgel films having compositions F-1 through F-4 were mounted onto a platform. A quarter inch ball probe traveled towards the softgel films at 2 mm/second until the probe penetrated the film. The measured force was the film's strength. The measured distance was the film's elasticity. The test was conducted at ambient conditions.

TABLE 3
Film Strength and Elasticity of Softgel
Shell compositions F-1 through F-4

| | F-1 | F-2 | F-3 | F-4 |
|---|---|---|---|---|
| Ingredient | Wt. % | Wt. % | Wt. % | Wt. % |
| Carrageenan | 2-20 | 2-15 | 2-15 | 2-15 |
| Plasticizer (e.g., sorbitol) | 8-45 | 8-45 | 8-45 | 8-45 |
| Sodium Phosphate Dibasic (Buffer | 0.2-4 | 0.2-4 | 0.2-4 | 0.2-4 |
| Agent) | | | | |
| Polyvinyl alcohol (PVA) | — | 0.5-12 | — | — |
| Pullulan (Water Soluble Polymer) | — | — | 1-15 | — |
| Polyvinyl alcohol-polyethylene | — | — | — | 1-20 |
| glycol copolymer (PVA-PEG | | | | |
| copolymer) (water soluble | | | | |
| polymer) | | | | |
| Film thickness (in) | 0.014 | 0.014 | 0.014 | 0.015 |
| Strength (Kg) | 4.9 | 5.8 | 5.5 | 5.6 |
| Elasticity (mm) | 7.5 | 8.3 | 8.7 | 7.7 |

As can be seen in Table 3, the control non-animal gelling agent capsule shell composition without a water soluble polymer had a strength of 4.9 kg and an elasticity of 7.5 mm. In comparison, each non-animal gelling agent capsule shell composition with a water soluble polymer (whether PVA by itself, Pullulan, or PVA-PEG copolymer), all were stronger, with a strength greater than 5 kg and more elastic with an elasticity greater than 8 mm.

### Example 3—Softgel Shell Compositions—Dissolution Data

Softgel shell compositions, prepared with varying concentrations and types of water soluble polymers, were cut into 1“×1” squares, and their solubility was evaluated in 0.1N HCl using USP Apparatus II with 75 RPM paddle speed in 0.1N HCl with deionized water at 37° C. Table 4 presents the solubility data (also referred to as dissolution data) on shell compositions prepared using various water-soluble polymers.

The type and concentrations of carrageenan, plasticizer, and buffer agent used in the samples of Table 4 below, were the same as described in Table 2 for formulations F-2 through F-6.

TABLE 4
Dissolution of Dried Softgel Shell composition
in 0.1N HCl Media at 37° C.

| | | Dissolution Time (Min) | |
|---|---|---|---|
| | | Dissolution | Complete Shell |
| | Concentration | Start | Dissolution |
| Polymer | Wt. % | Time (Min) | Time (Min) |
| Polyvinyl | 2 | 8 | 21 |
| Alcohol (PVA) | 4 | 8 | 22 |
| Pullulan | 2 | 6 | 27 |
| | 4 | 6 | 29 |
| | 5 | 9 | 28 |
| | 8 | 9 | 29 |

Without being construed as limiting, it is believed that the type of the water soluble polymer as well as the weight ratio of the carrageenan to the particular water soluble polymer may affect the dissolution time of the shell composition.

### Example 4—Softgel Capsule Formulations—Dissolution/Disintegration Data

Placebo softgel capsules using polyvinyl alcohol and pullulan were manufactured. The softgel capsules contained 900 mg of soybean oil as the fill material. The softgel shell compositions that encapsulated the fill material contained the type and concentrations of carrageenan, plasticizer, and buffer agent that were used in Table 2 for formulations F-2 through F-6. The water soluble polymer type and concentrations in the respective softgel fill materials that encapsulated the soybean oil were as described in Table 5. After the capsules were dried, the capsules were subjected to dissolution tests to evaluate the rupture time/solubility of capsules.

Dissolution Method:
Apparatus: USP Apparatus II
Temperature: 37° C.
Media: 0.1N HCl
Paddle speed: 50 and 75 RPM

TABLE 5
Dissolution of Placebo Non-Gelatin Softgel Capsules

| | | Sample Rupture Time (min) | | |
|---|---|---|---|---|
| | | Standard | 2 wt. % | 2 wt. |
| Dissolution condition | Observation | Control | Pullulan | % PVA |
| 0.1N HCl at 75 RPM | Shell Rupture | 16 | 3.5 | 13 |
| 37° C. | (min) | | | |
| 0.1N HCl at 50 RPM | Shell Rupture | 17 | 3.5 | — |
| 37° C. | (min) | | | |
| FASSGF** (Pepsin, | Shell Rupture | — | 3.3 | — |
| 50 RPM) 37° C. | (min) | | | |

**FASSSGF stands for Fasted-State Simulated Gastric Fluid

As can be seen from Table 5, softgel capsules with a shell composition that includes a water soluble polymer (PVA or Pullulan) exhibit faster dissolution time at 0.1N HCl at 37° C., at 75 RPM. The shell composition that included Pullulan had a faster dissolution time at 0.1N HCl than the control, and then that of the shell composition that included PVA. The shell composition that included Pullulan maintained a similar dissolution time at 0.1N HCl at 37° C. at 50 RPM and at 0.1N HCl with Pepsin at 37° C. at 50 RPM.

Disintegration tests were performed on the control sample, non-gelatin softgel capsules without pullulan, and on the non-gelatin softgel capsules containing 2 wt. % pullulan using USP disintegration apparatus. The result is presented in Table 6. Softgel capsules containing 2 wt. % pullulan ruptured much faster than the softgels without pullulan. The addition of pullulan resulted in faster disintegration.

TABLE 6
Disintegration Results of Non-Gelatin
Softgel Capsules Containing Pullulan

| | | Disintegration Time (min) | |
|---|---|---|---|
| | | Control (0 wt. % | 2 wt. % |
| Test condition | Observation | Pullulan) | Pullulan |
| Disintegration | Initial Rupture (min) | 16 | 7 |
| In 0.1N | Complete shell dissolution | 31 | 26 |
| HCl/37° C. | (min) | | |

### Example 5—Comparative Dissolution of Ibuprofen Softgel Capsules

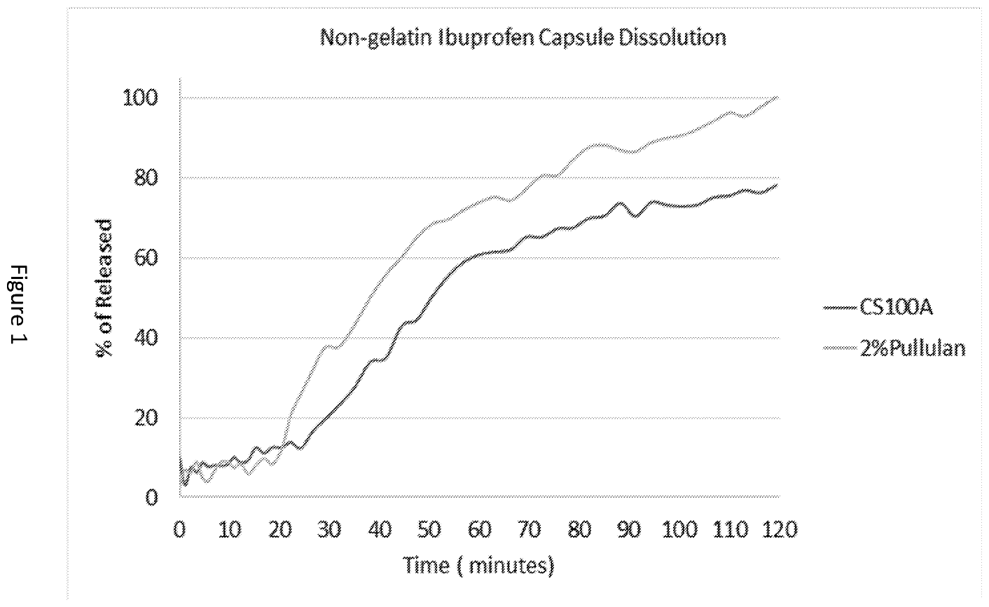

Comparative dissolution tests were conducted using non-gelatin softgel capsules containing 200 mg Ibuprofen manufactured using standard non-gelatin shell composition and non-gelatin shell composition, according to an embodiment, containing 2 wt. % pullulan. The softgel composition for the standard non-gelatin softgel capsule (CS100A) and for the non-gelatin softgel capsule containing 2 wt. % pullulan, according to an embodiment, are summarized in Table 7 below. FIG. 1 summarizes the outcome of the study. The dissolution test was performed on USP Apparatus II, paddle speed of 75 RPM, in 0.1N HCl at 37° C.

TABLE 7
Softgel Compositions for Ibuprofen Softgel Capsules

| | CS100A | 2 wt. % Pullulan |
|---|---|---|
| Ingredient | Wt. % | Wt. % |
| Carrageenan | 2-20 | 2-15 |
| Plasticizer | 8-45 | 8-45 |
| Sodium Phosphate (Buffering Agent) | 0.2-4 | 0.2-4 |
| Pullulan (Water Soluble Polymer) | — | 2-8 |

The result shows that non-gelatin Ibuprofen 200 mg softgel capsules containing 2 wt. % pullulan, according to an embodiment, ruptured sooner at 18 minutes than standard non-gelatin Ibuprofen 200 mg softgel capsules that ruptured at 24 minutes. Similarity Factor (F2) was calculated to be 40, which indicates that the two dissolution profiles are different. Pullulan softgel capsules had a faster drug release rate than standard control non-gelatin Ibuprofen 200 mg softgel capsules, which may help achieve a faster onset and shorter Tₘₐₓ.

The term “similarity factor (f2)” is a quantitative measure indicative of the similarity of the release profile of two dosage forms. The release profiles of two dosage forms are comparative, similar, or bioequivalent if the f2 value is no less than about 50 (based on the FDA Guidance to industry issued in December 2017 regarding bioequivalence studies for solid oral dosage forms).

The preceding description sets forth numerous specific details such as examples of specific systems, components, methods, and so forth, in order to provide a good understanding of several embodiments of the present invention. It will be apparent to one skilled in the art, however, that at least some embodiments of the present invention may be practiced without these specific details. In other instances, well-known components or methods are not described in detail in order to avoid unnecessarily obscuring the present invention. Thus, the specific details set forth are exemplary. Particular embodiments may vary from these exemplary details and still be contemplated to be within the scope of the present invention.

Although the operations of the methods herein are described in a particular order, the order of the operations of each method may be altered so that certain operations may be performed in an inverse order or so that certain operation may be performed, at least in part, concurrently with other operations. In another embodiment, instructions or sub-operations of distinct operations may be in an intermittent and/or alternating manner.

It is to be understood that the above description is intended to be illustrative, and not restrictive. Many other embodiments will be apparent to those of skill in the art upon reading and understanding the above description. The scope of the invention should, therefore, be determined with reference to the appended claims, along with the full scope of equivalents to which such claims are entitled.

1. A softgel composition comprising:
a shell composition comprising a non-animal derived gelling agent and a water soluble polymer, the shell composition completely dissolving in less than 30 minutes when subject to a dissolution with a USP Apparatus II with paddles at 75 RPM in 900 ml of 0.1N HCL and deionized water at 37 degrees C.; and
a fill material encapsulated within the shell composition.

2. The softgel composition of claim 1, wherein the non-animal derived gelling agent comprises carrageenan, starch, pregelatinized starch, xanthan gum, agar, pectin, alginate, sugar, high molecular weight polyethylene glycol, sugar derived alcohol, a cellulose derivative, a cellulosic polymer, hydroxyethylcellulose, hydroxypropylcellulose, hydroxypropylmethylcellulose, carboxymethylcellulose, microcrystalline cellulose, attapulgite, bentonite, dextrin, alginate, kaolin, lecithin, magnesium aluminum silicate, carbomer, carbopol, silicon dioxide, curdlan, furcelleran, albumin, soy protein, chitosan, or a combination thereof.

3. The softgel composition of claim 1, wherein the shell composition further comprises a plasticizer.

4. (canceled)

5. The softgel composition of claim 1, wherein the water soluble polymer comprises polyvinyl alcohol, pullulan gum, polylactic acid, polyvinyl alcohol-polyethylene glycol graft co-polymer, high molecular weight polyethylene glycol, povidone, a surfactant, or a combination thereof.

6. (canceled)

7. (canceled)

8. The softgel composition of claim 1, wherein the non-animal derived gelling agent comprises carrageenan and starch.

9. The softgel composition of claim 8, wherein the weight ratio of carrageenan to starch in the shell composition is about 1:1 to about 1:10.

10. (canceled)

11. (canceled)

12. The softgel composition of claim 1, wherein the non-animal gelling agent is in an amount of from about 2 wt. % to about 40 wt. %, based on total weight of the softgel capsule shell composition.

13. The softgel composition of claim 1, comprising less than 10 wt. % of an animal derived gelling agent, based on total weight of the softgel capsule shell composition.

14. The softgel composition of claim 1, wherein the shell composition does not contain an animal derived gelling agent.

15. (canceled)

16. The softgel composition of claim 2, comprising the high molecular weight polyethylene glycol, wherein the high molecular weight polyethylene glycol has a range of number average molecular weight of 600 Da-2,000,000 Da or a combination of polyethylene glycols of varying number average molecular weights within the range of 600 Da-2,000,000 Da.

17. The softgel composition of claim 3, wherein the plasticizer is in the softgel capsule shell composition in an amount of from about 0.5 wt. % to about 40 wt. %, based on total weight of the softgel capsule shell composition.

18. (canceled)

19. (canceled)

20. A softgel composition comprising
a fill material comprising an active agent or a cosmetic agent, wherein the fill material is encapsulated by a shell composition, and wherein the shell composition comprises a non-animal derived gelling agent and a water soluble polymer, the shell composition completely dissolving in less than 30 minutes when subject to a dissolution with a USP Apparatus II with paddles at 75 RPM in 900 ml of 0.1N HCL and deionized water at 37 degrees C.

21. A softgel composition comprising a fill material encapsulated by a shell composition, wherein the shell composition comprises:
a non-animal derived gelling agent comprising carrageenan, starch, or a combination thereof; and
a water soluble polymer comprising polyvinyl alcohol, pullulan gum, polylactic acid, polyvinyl alcohol-polyethylene glycol graft co-polymer, high molecular weight polyethylene glycol, povidone, a surfactant, or a combination thereof.

22. The softgel composition of claim 20, wherein the fill material comprises a hydrophilic material, a lipophilic, an amphiphilic material or a combination thereof.

23. (canceled)

24. (canceled)

25. (canceled)

26. A method of preparing a softgel composition comprising:
combining a non-animal gelling agent, a water soluble polymer, and optionally at least one of a buffering agent, a plasticizer, or water to form a combination; and
encapsulating a fill material in a shell composition comprising the combination.

27. The method of claim 26, wherein combining comprises:
mixing the plasticizer with water to form a plasticizer solution; and
mixing the water soluble polymer, non-animal gelling agent, and the buffering agent with the plasticizer solution to form the combination.

28. The method of claim 26, wherein combining comprises:
mixing the plasticizer, the non-animal gelling agent, and the water to form a plasticizer solution;
mixing the water soluble polymer and the buffering agent with the plasticizer solution to form the combination.

29. The method of claim 26, further comprising heating the combination to form a molten mass.

30. The method of claim 29, wherein the molten mass is uniform.

31. (canceled)

32. (canceled)

33. A method of treating a condition, comprising administering to a subject in need thereof the softgel composition of claim 1, wherein the condition is treated, prevented, ameliorated, or reduced upon administration.
